SEQUENCE LISTING (16 sequences)

SEQ ID NO: 1 - DNA, 297 bp, Artificial Sequence
  Description of Artificial Sequence Synthetic polynucleotide
gacacgctgaattcacaacacaaatcagatttatagagagatttataaaaaaaaaaaaacatgtctcgagctcggatcctctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacc

SEQ ID NO: 2 - DNA, 6153 bp, Artificial Sequence
  Description of Artificial Sequence Synthetic polynucleotide
aattccgcccctagaaatatttgcgactcttctggcatgtaatatttcgttaaatatgaagtgctccatttttattaactttaaataattggttgtacgatcactttcttatcaagcgttactaaaatgcgtcaatctctttgttcttccatattcatatgtcaaaatctatcaaaattcttatatatctttttcgaatttgaagtgaaatttcgataatttaaaattaaatagaacatatcattatttaggtgtcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggcgcggggcgcgcccggtccaaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaattcacaacacaaatcagatttatagagagatttataaaaaaaaaaaaacatgtctcgagctcggatcctctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccgggcggccggccgcccctagaaatatttgcgactcttctggcatgtaatatttcgttaaatatgaagtgctccatttttattaactttaaataattggttgtacgatcactttcttatcaagcgttactaaaatgcgtcaatctctttgttcttccatattcatatgtcaaaatctatcaaaattcttatatatctttttcgaatttgaagtgaaatttcgataatttaaaattaaatagaacatatcattatttaggtgtcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggcgggtggagggggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgacaagctacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggactctagctagaggatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgatcgaatactaacgtctctaccacattgaagagcaagct

SEQ ID NO: 3 - DNA, 7564 bp, Artificial Sequence
  Description of Artificial Sequence Synthetic polynucleotide
aattccgcccctagaaatatttgcgactcttctggcatgtaatatttcgttaaatatgaagtgctccatttttattaactttaaataattggttgtacgatcactttcttatcaagcgttactaaaatgcgtcaatctctttgttcttccatattcatatgtcaaaatctatcaaaattcttatatatctttttcgaatttgaagtgaaatttcgataatttaaaattaaatagaacatatcattatttaggtgtcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggcgcggggcgcgcccggtccaaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaattcacaacacaaatcagatttatagagagatttataaaaaaaaaaaaacatgtctcgagctcggatcctctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccgggcggccggccgcccctagaaatatttgcgactcttctggcatgtaatatttcgttaaatatgaagtgctccatttttattaactttaaataattggttgtacgatcactttcttatcaagcgttactaaaatgcgtcaatctctttgttcttccatattcatatgtcaaaatctatcaaaattcttatatatctttttcgaatttgaagtgaaatttcgataatttaaaattaaatagaacatatcattatttaggtgtcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggcgggtggagggggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgacaagctacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggactctagctagaggatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgatcgaatactaacgtctctaccagatatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagttccttcccgcttcagtgacaacgtcgagcacagctgcgcaaggaacgcccgttgtggccagccacgatagccgcgctgcctcgtcttggagttcattcagggcaccggacaggtcggtcttgacaaaaagaaccgggcgcccctgcgctgacagccggaacacggcggcatcagagcagccgattgtttgttgtgcccagtcatagccgaatagcctctccacccaagcggccggagaacctgcgtgcaatccatcttgttcaatcatgcctgcatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataacgtgactcccttaattctccgctcatgatcgatatccattgaagagcaagct

SEQ ID NO: 4 - DNA, 50 bp, Artificial Sequence
  Description of Artificial Sequence Synthetic oligonucleotide
ttgtcattaattaacggcctagggcggccgcggtcctgcaacttgaggta

SEQ ID NO: 5 - DNA, 404 bp, Arabidopsis thaliana
caaacacacgctcggacgcatattacacatgttcatacacttaatactcgctgttttgaattgatgttttaggaatatatatgtagagagagcttccttgagtccattcacaggtcgtgatatgattcaattagcttccgactcattcatccaaataccgagtcgccaaaattcaaactagactcgttaaatgaatgaatgatgcggtagacaaattggatcattgattctctttgattggactgaagggagctccctctctcttttgtattccaattttcttgattaatctttcctgcacaaaaacatgcttgatccactaagtgacatatatgctgccttcgtatatacagttctggtaaaattaacattttgggtttatctttatttaaggcatcgccatg

SEQ ID NO: 6 - DNA, 21 bp, Homo sapiens
cacccagctaagagactgcct

SEQ ID NO: 7 - DNA, 21 bp, Homo sapiens
tggcagtgtcttagctggttg

SEQ ID NO: 8 - DNA, 415 bp, Artificial Sequence
  Description of Artificial Sequence Synthetic polynucleotide
gaattcaaacacacgctcggacgcatattacacatgttcatacacttaatactcgctgttttgaattgatgttttaggaatatatatgtagcacccagctaagagactgccttcacaggtcgtgatatgattcaattagcttccgactcattcatccaaataccgagtcgccaaaattcaaactagactcgttaaatgaatgaatgatgcggtagacaaattggatcattgattctctttgatggcagtgtcttagctggttgctctcttttgtattccaattttcttgattaatctttcctgcacaaaaacatgcttgatccactaagtgacatatatgctgccttcgtatatatagttctggtaaaattaacattttgggtttatctttatttaaggcatcgccatgtctaga

SEQ ID NO: 9 - DNA, 21 bp, Homo sapiens
tgagatgaagcactgtagctc

SEQ ID NO: 10 - DNA, 21 bp, Homo sapiens
gaactacagtgctacatctct

SEQ ID NO: 11 - DNA, 415 bp, Artificial Sequence
  Description of Artificial Sequence Synthetic polynucleotide
gaattcaaacacacgctcggacgcatattacacatgttcatacacttaatactcgctgttttgaattgatgttttaggaatatatatgtaggaactacagtgctacatctcttcacaggtcgtgatatgattcaattagcttccgactcattcatccaaataccgagtcgccaaaattcaaactagactcgttaaatgaatgaatgatgcggtagacaaattggatcattgattctctttgatgagatgaagcactgtagctcctctcttttgtattccaattttcttgattaatctttcctgcacaaaaacatgcttgatccactaagtgacatatatgctgccttcgtatatatagttctggtaaaattaacattttgggtttatctttatttaaggcatcgccatgtctaga

SEQ ID NO: 12 - DNA, 23 bp, Homo sapiens
gtccagttttcccaggaatccct

SEQ ID NO: 13 - DNA, 21 bp, Homo sapiens
agggtcctgggaatactggag

SEQ ID NO: 14 - DNA, 417 bp, Artificial Sequence
  Description of Artificial Sequence Synthetic polynucleotide
gaattcaaacacacgctcggacgcatattacacatgttcatacacttaatactcgctgttttgaattgatgttttaggaatatatatgtagagggtcctgggaatactggagtcacaggtcgtgatatgattcaattagcttccgactcattcatccaaataccgagtcgccaaaattcaaactagactcgttaaatgaatgaatgatgcggtagacaaattggatcattgattctctttgagtccagttttcccaggaatccctctctcttttgtattccaattttcttgattaatctttcctgcacaaaaacatgcttgatccactaagtgacatatatgctgccttcgtatatatagttctggtaaaattaacattttgggtttatctttatttaaggcatcgccatgtctaga

SEQ ID NO: 15 - protein, 6 aa, Artificial Sequence
  Description of Artificial Sequence Synthetic 6xHis tag
HHHHHH

SEQ ID NO: 16 - protein, 6 aa, Unknown
  Description of Unknown Endoplasmic reticulum retention peptide
SEKDEL

(19) United States
(12) Patent Application Publication
(10) Pub. No.: US 2017/0246321 A1
(43) Pub. Date: Aug. 31, 2017

(54) Edible Plants Producing Bioengineered Micro-RNA for Gene Regulation Upon Ingestion

(71) Applicant: University of South Carolina, Columbia, SC (US)

(72) Inventors: Vicki Vance, Columbia, SC (US); Keith Lynn Wycoff, Palo Alto, CA (US)

(21) Appl. No.: 15/443,279

(22) Filed: Feb. 27, 2017

Related U.S. Application Data
(60) Provisional application No. 62/300,239, filed on Feb. 26, 2016.
(60) Provisional application No. 62/498,758, filed on Jan. 5, 2017.

(51) Int. Cl. A61K 48/00; A61K 9/00; C12N 15/113

(52) U.S. Cl. CPC A61K 48/0066; C12N 15/113; C12N 15/1135; C12N 15/1138; A61K 9/0053; C12N 2800/107

(57) ABSTRACT

Methods and materials providing a route for the use of transgenic plants as bio-factories to produce therapeutic miRNAs are described. The plants can be ingestible and can be used to deliver to a subject in need thereof a therapeutic miRNA by ingestion of the bioengineered plant tissue that carries an exogenous genetic sequence for the therapeutic miRNA. The therapeutic miRNA can be useful in treating a disease state such as cancer. The therapeutic miRNA can be a mammalian tumor suppressor miRNA.

### CROSS-REFERENCE TO RELATED APPLICATION

This application claims filing benefit of U.S. Provisional Patent Application Ser. No. 62/300,239 having a filing date of Feb. 26, 2016 entitled “Edible Plants Producing Bioengineered Micro-RNA for Gene Regulation Upon Digestion,” and to U.S. Provisional Patent Application Ser. No. 62/498,758 having a filing date of Jan. 5, 2017, entitled “miRNA Orally Dosage Forms,” both of which being incorporated herein by reference for all purposes.

### STATEMENT REGARDING FEDERALLY SPONSORED RESEARCH OR DEVELOPMENT

This invention was made with government support under grant #IOS-1029803 awarded by the National Science Foundation and under grant #CA181895-01A1 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 21, 2017, is named USC-513_SL.txt and is 23,357 bytes in size.

### BACKGROUND

MicroRNAs (miRNAs) are a class of small non-coding RNAs that play a critical role in gene expression in nearly all eukaryotic organisms. These small RNAs (˜21-24 nucleotides long) work by incorporating into a protein complex called RISC where the miRNAs act as guides to find target messenger RNAs (mRNAs) based on complementarity. Binding of RISC to the target RNA via the miRNA causes degradation of the target message or blocks translation, in either case preventing production of the encoded protein. Accordingly, miRNAs are considered master regulators of the genome. Each organism produces hundreds to several thousands of miRNAs, each of which is thought to control expression of many protein-coding genes, thereby regulating virtually every physiological process in the body. It has become clear that reduced accumulation of some miRNAs and increased accumulation of others is associated with many mammalian diseases. In a number of cell culture or animal model systems, restoration of the miRNA balance has been shown to block disease progression, and the therapeutic potential of miRNAs is now widely accepted.

Misregulation of miRNA production associated with disease has been studied in a variety of diseases including cancers. Typically, tumor cells show a decrease in the level of certain miRNAs called tumor suppressor miRNAs. Although the exact mechanism of action of tumor suppressor miRNAs is not known, it is clear that the overall impact of reduced levels of these small RNAs is to promote one or more aspects of tumorigenesis. In several cases, experiments using animal models have demonstrated that loss of a particular tumor suppressor miRNA plays a causative role in tumor initiation, progression and/or spread. In these cases, tumorigenesis can be suppressed when the missing miRNA is added back. Thus, restoring the levels of a missing or reduced tumor suppressor miRNA appears quite promising for treating cancer; however, as is the case for other therapies based on small RNAs, delivery of the miRNA to the diseased tissue is a critical barrier to implementation.

As a result, treatments utilized today center around conventional therapies that are generally invasive and/or carry the risk of substantial side effects. For instance, conventional treatment for patients with chronic life-threatening bowel disease such as colorectal cancers consists of oral NSAIDs combined with invasive surveillance with excision and pathological evaluation of intestinal polyps. Despite improvements in conventional therapies, particularly due to increased screening that results in reduced mortality by detecting tumors at earlier stages when they are more successfully treated by surgery, problems still exist with conventional approaches to such diseases. For instance, many people are reluctant to be screened because it is both invasive and expensive. In addition, recurrence of cancer following surgery is a major problem, and many patients ultimately die from the disease.

miRNA-based technologies for such diseases have been developed that may compete with traditional therapies. Unfortunately, these methods also present patient response issues as they involve invasive parenteral routes of administration including subcutaneous, intramuscular and intravenous.

One non-traditional approach for disease treatment that has been labeled “alternative” and less acceptable with the medical industry is ingestion of plants that are understood to carry beneficial compounds. Upon successful ingestion and uptake of these compounds into the system, they can function as molecular therapies for a disease state. It has long been known that ingested RNA from food sources can be taken up by the digestive system of both nematodes and insects, and can control the expression of genes in those organisms. In 2012, the first evidence that a similar phenomenon occurs in mammals was reported. The authors showed that miRNAs from plant-based foods are taken up by cells of the mammalian gastrointestinal (GI) tract and are capable of regulating the expression of mammalian genes. The concept generated a good deal of excitement in the scientific community, but also met with some skepticism. Whereas some follow-up studies reported uptake of plant-based dietary small RNAs in mammals, others failed to detect such RNAs in response to various feeding regimens. Moreover, these approaches are limited to the natural compounds present in the plants, which are often unproven as molecular therapies.

Accordingly, what is needed in the art is an effective delivery system for proven therapeutic miRNAs. Moreover, an oral delivery system that can avoid issues known for more traditional, invasive routes would be highly beneficial.

### SUMMARY

According to one embodiment, disclosed is a cell that includes an exogenous genetic sequence. The exogenous genetic sequence encodes a therapeutic miRNA that modulates a target nucleotide sequence. Additionally, the cell that includes the exogenous genetic sequence is free of the target nucleotide sequence.

According to another embodiment, disclosed is a method for forming an ingestible therapeutic composition. The method includes forming a modified miRNA precursor gene to include a genetic sequence encoding a therapeutic miRNA that modulates a target nucleotide sequence and introducing the modified miRNA precursor gene into a cell that is free of the target nucleotide sequence. In addition, the genetic sequence that encodes the therapeutic miRNA is exogenous to the cell. The method also includes generating plant tissue that includes the cell and/or the therapeutic miRNA expression product of the genetic sequence and incorporating the plant tissue into an ingestible therapeutic composition.

Also disclosed is a method for delivery of a therapeutic miRNA to a subject in need thereof. The method can include providing to the subject an ingestible composition that includes tissue of a plant. The plant can include a genetic sequence that encodes the therapeutic miRNA, and the genetic sequence can be exogenous to the plant. The plant tissue of the ingestible composition can include the genetic sequence and/or the therapeutic miRNA expression product of the genetic sequence. The therapeutic miRNA can modulate a target nucleotide sequence that is absent from the plant and that is carried by the subject.

### BRIEF DESCRIPTION OF THE DRAWINGS

A full and enabling disclosure, including the best mode thereof, to one of ordinary skill in the art, is set forth more particularly in the remainder of the specification, including reference to the accompanying figures, in which:

FIG. 1 illustrates a pTRAc vector and two derivatives of the pTRAc vector as may be utilized in forming an expression cassette as described herein. FIG. 1 discloses “His6” as SEQ ID NO: 15 and “SEKDEL” as SEQ ID NO: 16.

FIG. 2 illustrates a TRBO vector as may be utilized in forming an expression cassette as described herein.

FIG. 3A illustrates the reduction in tumor burden in miRNA-treated compared to water-treated mice. The table shows the number of tumors in each mouse in the three groups listed in ascending order within each group: miRNA=tumor suppressor miRNA cocktail+total plant RNA; RNA=total plant RNA alone; water=only water. The number of tumors from mice in the miRNA-treated and water-treated groups were plotted to show the distributions that are the basis of the K-S statistical analysis.

FIG. 3B presents the mean number of tumors in treatment groups described in the Examples section. Error bars show the standard error of the mean.

FIG. 4 presents the results of RT-qPCR after oxidation, showing higher relative miR-34a levels in miRNA-treated versus water-treated mice. The mean relative miR-34a and miR-100 concentrations are shown. Error bars are the standard error of the mean.

FIG. 5 provides gel blot analysis of RNA samples isolated from wild type (WT) Arabidopsis and homozygous lines expressing miR-34a, miR-143 or miR-145. The left panel illustrates the results following probe for all three tumor suppressor miRNAs. In the right panel, a duplicate WT sample run on the same gel and blotted together was probed for the highly expressed endogenous plant miRNA, miR168a. The ethidium bromide stained gel is shown as loading control below the autoradiograms.

FIG. 6 illustrates MiR168a in RNA samples isolated from fresh (first two lanes) or lyophilized (last two lanes) plant tissue. EtBr staining is shown as a loading control.

FIG. 7 illustrates results following isolation of 10 μg of RNA from exosomes and loading thereof into each of four lanes of a small RNA gel. The resulting blot was cut to separate the four lanes, and each was hybridized separately for miR-34a, mir-143, miR-145, or the endogenous plant miR159a.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, disclosed herein are methods and materials providing a route for the use of transgenic plants as bio-factories to produce therapeutic miRNAs. Beneficially, the plants can be ingestible and can be used to deliver to a subject in need thereof a therapeutic miRNA by ingestion of the bioengineered plant tissue. For instance, a method can utilize a natural plant miRNA precursor gene as a starting structure, and the endogenous miRNA encoding sequence of the precursor gene can be replaced with an exogenous miRNA sequence that encodes a therapeutic miRNA that targets a genetic sequence of interest. In particular, the intended therapeutic target of the miRNA is not carried by the transformed plant cell. It should be understood that while a plant cell may carry off-target sequences that may inadvertently interact with the miRNA expression product of the exogenous sequence, such sequences are not the intended target, and the intended target is not present in the modified plant cell that carries the exogenous miRNA sequence. A method can also replace the endogenous microRNA* sequence of the precursor gene to maintain the secondary structure (e.g., mismatches) of the starting precursor expression product and insure proper processing by the cell to produce the exogenous miRNA product.

Unlike mRNA, rRNA, and tRNA, which are degraded within seconds after being placed in a nuclease-rich extracellular environment, biologically derived (as opposed to synthesized or purified) miRNAs survive under unfavorable physiological conditions such as extreme variations in pH and are actually quite stable and abundant in circulation. There are two reasons for this: 1) in vivo, miRNAs are stabilized by binding to Argonaute proteins, and 2) miRNAs are selectively encapsulated into exosomes. Exosomes are 40-100 nm diameter membranous vesicles that are released from all eukaryotic cells. They carry a cargo of proteins, lipids, mRNAs, and/or miRNAs that they can transfer to recipient cells, thus serving as extracellular messengers mediating cell-cell communication.

As is known in the art, miRNA precursors have a stem-loop structure with the miRNA sequence located within the stem. The precursor is processed by DICER-LIKE1 (DCL1) to produce a small duplex with one strand of the duplex being the mature miRNA, and the other strand of the duplex designated miRNA*. During use, the miRNA incorporates into the RISC complex where it acts as a guide to direct RISC to target mRNA by the base pairing rules. The targeted mRNAs are either degraded or their translation is inhibited. The miRNA* strand is rapidly degraded.

Following formation, a modified miRNA precursor gene including an exogenous miRNA encoding sequence can be cloned into a suitable expression vector and introduced into a cell that can then be utilized in development of ingestible plant tissue. A therapeutic composition can then be formulated from the plant tissue. In particular, the therapeutic composition can include the modified cell and/or the miRNA expressed by the cell for delivery to a subject in need thereof. Upon ingestion of the therapeutic composition, the miRNA expression product of the modified miRNA precursor gene can be delivered to the subject via the natural digestion and molecular pathways of the subject. As specific dietary parameters and the health of the GI tract can have a dramatic impact on the uptake of dietary components, the means of delivery as well as other components of an ingestible composition can be varied as needed. For instance, an ingestible composition can be provided as a solid or a liquid and can be delivered via eating, drinking or directly into the stomach, duodenum or jejunum via enteral feeding.

According to the present disclosure, a method can include modification of a starting miRNA precursor genetic sequence by replacement of the endogenous miRNA encoding sequence with an exogenous miRNA encoding sequence of interest and replacement of the corresponding endogenous miRNA* sequence with a designed miRNA* sequence to maintain the secondary structure (e.g., bulges, etc.) of the original endogenous miRNA/miRNA* duplex so as to maintain the structure of the precursor to ensure proper processing. As utilized herein, the term “endogenous” is intended to refer to a sequence that is natively present in the genome of an organism and the term “exogenous” is intended to refer to a sequence that is not natively present in an organism.

In one embodiment, the starting miRNA precursor gene can be an endogenous gene of the cell, e.g., a plant cell, that will be incorporated in the plant that is intended for use in forming the ingestible therapeutic composition, but it should be understood that this is not a requirement of the disclosure and in other embodiments the starting miRNA precursor gene can be endogenous to a different organism. In this case, the miRNA encoding sequence can be exogenous to both the organism of the starting miRNA precursor gene and the plant to which the modified miRNA precursor gene will be introduced. In any case, the modified miRNA precursor gene that includes the exogenous miRNA encoding sequence can be configured for transcription within an edible plant such that the therapeutic miRNA can be expressed by the plant.

The starting miRNA precursor gene can be modified to include the exogenous miRNA sequence according to standard methodology as is known in the art. In one embodiment, the modified miRNA precursor gene can be constructed by obtaining the sequence of a known miRNA precursor gene and replacing the endogenous miRNA encoding sequences therein with the miRNA encoding sequences directed to the target of interest and replacement of the endogenous miRNA* sequence with an miRNA* sequence such that the expressed exogenous miRNA/miRNA* duplex maintains the secondary structure of the endogenous miRNA/miRNA* duplex for proper processing. Methods for constructing modified precursor miRNAs are known in the art, and thus are not described at length herein.

The miRNA that is encoded by the exogenous sequence of the modified miRNA precursor gene can provide direct or indirect therapeutic functionality through modulation of a target. For instance, the product miRNA of the exogenous sequence can modulate a target sequence that is directly involved in a disease process, e.g., tumorigenesis, or can modulate a target sequence that is indirectly involved in a disease process, e.g., a gene encoding a protein that resides within a functional pathway of a disease process or a disease symptom mitigation pathway.

The miRNA can be complementary to a target RNA of interest. While the miRNA can be completely complementary to a target RNA, in one embodiment mismatches may be tolerated. For instance from 1 to about 6 nucleotide mismatches may occur between the miRNA and the target miRNA, or from about 2 to about 3 mismatched nucleotides in some embodiments. While the mismatched nucleotides may occur throughout the miRNA sequence, in one embodiment they can be located near the center of the molecule. In animals, complementarity of miRNA to target is highly dependent on the seed region of the miRNA. That region is at or near the 5′ end of the miRNA and can include about 10 nucleotides that are completely complementary to the target. The 3′ end of the miRNA can generally tolerate more mismatches.

The miRNA can be fully or partially complementary to any region of a target RNA including the 3′ untranslated region, coding region, etc. In this manner, the miRNA can be utilized to regulate a specific target gene by any mechanism of action. For instance, the miRNA can alter the production, processing, stability, or translation of the target RNA. The miRNA can generally be a relatively small molecule comprising about 15 to about 30 nucleotides, about 20 to about 28 nucleotides, or specifically about 21 to about 24 nucleotides in some embodiments.

The modified miRNA precursor expression product can be designed to mimic endogenous miRNA precursors. As with an endogenous miRNA, the single-stranded therapeutic miRNA can be processed from the stem of the precursor and can be flanked by bulges. The miRNA precursor can have all the features of an endogenous miRNA precursor, but the position of the endogenous miRNA can be replaced with that of the desired therapeutic miRNA. For example, the expressed miRNA precursor can contain a loop chosen from an endogenous miRNA precursor. The therapeutic miRNA can include a sequence that is complementary to the target miRNA sequence, e.g., complementary to either the 3′-untranslated region or the coding region, and the coding sequence for the therapeutic miRNA can be inserted in a position in the modified precursor miRNA gene analogous to the endogenous miRNA coding sequence within the gene. Bulges can be included and these too can be taken from the context of the endogenous precursor in some embodiments.

In one embodiment, the therapeutic miRNA can target an animal or human nucleotide sequence that is directly or indirectly involved in a disease process. For instance, the therapeutic miRNA can be effective in cancer treatment. By way of example, the modified miRNA precursor gene can express one or more mammalian tumor suppressor miRNAs including, without limitation, miR-34a, miR-143, or miR-145. These three miRNAs are all down-regulated in colorectal cancer cells, and that down-regulation is associated with mutations in the Apc gene. Downregulation of miR-143 seems to have a role in initiation of colorectal cancer, while downregulation of miR34a may be more involved in acquiring the ability to spread and invade other organs. Downregulation of both miR-143 and miR-145 appears to occur early in cancer development, while miR-34 is turned off later. Replacement of miR-143 and -145 inhibits growth of CRC in vivo. Overexpression of miR-143, along with miR-145, reduces the development of tumors in Apc^{Min/+} mice. Restoration of miR-34 has also been shown to reduce colorectal cancer cell growth.

The target of the miRNA can include any nucleotide sequence of interest, including genes, regulatory sequences, etc. Genes of interest include those associated with any disease state include cancer, congenital diseases, viral diseases, bacterial diseases, metabolic disorders, chronic or acute inflammatory conditions, fibrotic conditions such as hepatic, pulmonary or dermal fibrosis etc. In one particular embodiment, the method can be utilized in colon cancer prevention or treatment. People suffering from inflammatory bowel disease or familial adenomatous polyposis (FAP) are also at high risk of developing colon cancer. Accordingly, a method can be directed to treatment or prevention of inflammatory bowel disease or FAP. In some embodiments, the method can be utilized to target infectious diseases caused by viruses. For example, an anti-viral miRNA can be designed to target viral RNA of a subject.

The genes may be involved in metabolism of oil, starch, carbohydrates, nutrients, chemotherapies, etc. Target sequences also include genes responsible for the synthesis of proteins, peptides, fatty acids, lipids, waxes, oils, starches, sugars, carbohydrates, flavors, odors, toxins, carotenoids, hormones, polymers, flavonoids, glycoproteins, glycolipids, etc. Targets can include, without limitation, RNA sequences involved in production of proteins, hormones (e.g., insulin, human growth hormone, interferons, etc.), cytokines, serum albumin, hemoglobin, collagen, etc.

The methods can be used to target and thereby decrease the expression of any gene or sequence of interest including therapeutic or immunogenic peptides and proteins, nucleic acids for controlling gene expression, genes to reproduce enzymatic pathways for chemical synthesis, genes to shunt an enzymatic pathway for expression of a particular intermediate or final product, and the like. The target sequence may include those that are altered in various ways due to a disease process including, without limitation, amino acid substitutions, deletions, truncations, and insertions. For instance, in one embodiment, a disease state can be caused by targeted messages producing too much of their encoded proteins, and treatment by use of an miRNA as described herein can correct that by decreasing the level of target gene expression.

In one embodiment, a target can be modulated by the miRNA so as to produce peptides or proteins that cannot effectively be produced by the natural gene expression systems of the subject. This lack of production can be, e.g., due to the disease state that is being treated. For example, delivery of the miRNA to a subject can directly or indirectly lead to increase in expression of a protein involved in cell viability, cell proliferation, cellular differentiation, or protein assembly and can thereby mitigate the effects of a disease state. In one embodiment, upon delivery of the miRNA to the target the subject can exhibit increase production of one or more regulatory proteins.

A target sequence encompassed herein can also include fragments and variants of proteins or regulator sequences. The term “fragment” as utilized herein is intended to refer to a portion of an endogenous nucleotide sequence. A target that is a fragment of an endogenous sequence may or may not retain biological activity. Such targeting sequences may be useful as hybridization probes, as antisense constructs, or as co-suppression sequences. Thus, fragments of a nucleotide sequence may include those of about 20 nucleotides or more, about 50 nucleotides or more, about 100 nucleotides or more, as well as full-length endogenous nucleotide sequences.

As utilized herein, the term “variant” is intended to refer to substantially similar sequences. Nucleotide sequences can be considered to be substantially similar if the sequences hybridize to each other under stringent conditions. Generally, stringent conditions can be about 5° C. lower than the thermal melting point for the specific sequence at a defined ionic strength and pH. Typically, stringent conditions can be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary stringent conditions include a buffer solution of 30 to 35% formamide, 1.0 M NaCI, 1% SDS (sodium dodecyl sulfate) at 37° C., and a wash in 1× to 2×SSC (20×SSC=3.0 M NaCl/0.3 M trisodium citrate) at 50 to 55° C. It is recognized that the temperature salt and wash conditions may be altered to increase or decrease stringency conditions. The duration of hybridization is generally less than about 24 hours, usually about 4 to about 12 hours. For the post-hybridization washes, the critical factors are the ionic strength and temperature of the final wash solution.

Conservative nucleotide variants include those sequences that, because of the degeneracy of the genetic code, encode the amino acid sequence of the subject sequence. Conservative variants need not hybridize to each other under stringent conditions but may still be considered to be substantially similar if the polypeptides they encode are substantially identical, for example when a copy of a nucleic acid is created using the maximum codon degeneracy permitted by the genetic code.

Variant nucleotide sequences can include synthetically derived sequences, such as those generated, for example, using site-directed mutagenesis.

When considering proteinaceous sequences, a variant can include a protein derived from the endogenous protein by deletion or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or human manipulation. Conservative amino acid substitutions will generally result in variants that retain biological function.

Variant proteins can be biologically active, that is they continue to possess the desired biological activity of the native protein. A biologically active variant of a protein may differ from that protein by as few as 1 to about 15 amino acid residues, 1 to about 10, 6 to about 10, as few as 5, as few as 4, 3, 2, or even 1 amino acid residue.

The modified miRNA precursor gene that includes an exogenous genetic sequence encoding a therapeutic miRNA can be inserted into a plant cell for expression, for instance through formation of an expression cassette including the modified miRNA precursor gene and insertion into the plant of interest by use of a suitable vector.

In general, an expression cassette can include 5′ and 3′ regulatory sequences operably linked to the miRNA precursor nucleotide sequence. By “operably linked” is intended a functional linkage between sequences, e.g., between a promoter sequence and a second sequence wherein the promoter sequence initiates and mediates transcription of the second sequence. Generally, operably linked means that the nucleic acid sequences that are linked are contiguous and, where necessary to join two protein coding regions, contiguous and in the same reading frame.

The expression cassette can include in the 5′-3′ direction of transcription, a transcriptional and translational initiation region, a DNA sequence including the miRNA precursor gene and a transcriptional and translational termination region functional in plants. The transcriptional initiation region that includes the promoter may be a native promoter, a variant of the native promoter, or heterologous to the plant cell. Additionally, the promoter may be a natural promoter sequence or a synthetic sequence. As utilized herein, the term “heterologous” is intended to refer to a sequence that is not found in the native plant into which the sequence is introduced. In those embodiments in which the promoter is heterologous to the native plant cell, the expression cassette can be termed as incorporating a chimeric gene, which includes a coding sequence operably linked to a transcription initiation region that is heterologous to the coding sequence.

A termination region may be native to the same organism as that from which the transcriptional initiation region is derived, may be native to the same organism as that from which the starting miRNA precursor gene is derive, may be native to the same organism as that from which the exogenous miRNA encoding sequence is derived, or may be derived from another source. By way of example, convenient termination regions are available from the Ti-plasmid of A. tumefaciens, such as the octopine synthase and nopaline synthase termination regions.

Any number of promoters can be used in an expression cassette. For instance, constitutive, tissue-preferred, inducible, developmental, or other promoters for expression in plants can be utilized. A preferred promoter can be selected depending upon specific characteristics of the system such as the specific plant cell that will incorporate the expression cassette, the exogenous miRNA to be expressed by the system, the level of expression desired, etc.

Constitutive promoters include, for example, CaMV 35S promoter; ubiquitin; MAS; ALS promoter, and the like. Other constitutive promoters include those in U.S. Pat. Nos. 5,608,149; 5,608,144; 5,604,121; 5,569,597; 5,466,785; 5,399,680; 5,268,463; and 5,608,142, all of which are incorporated herein by reference.

A number of inducible promoters are known in the art. For resistance genes, a pathogen-inducible promoter can be utilized. Such promoters include those from pathogenesis-related proteins (PR proteins), which are induced following infection by a pathogen; e.g., PR proteins, SAR proteins, beta-1,3-glucanase, chitinase, etc. Inducible promoters can include promoters that are expressed locally at or near the site of vector infection. A wound-inducible promoter may be used in the constructs. Such wound-inducible promoters include potato proteinase inhibitor (pin II) gene; wun1 and wun2, U.S. Pat. No. 5,428,148 (incorporated herein by reference); win1 and win2; system in; WIPI; MPI gene; and the like.

Chemical-regulated promoters can be used to modulate the expression of the miRNA in a plant through the application of an exogenous chemical regulator. Depending upon the objective, the promoter may be a chemical-inducible promoter, where application of the chemical induces expression, or a chemical-repressible promoter, where application of the chemical represses expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid. Other chemical-regulated promoters of interest include steroid-responsive promoters (e.g., the glucocorticoid-inducible promoter) and tetracycline-inducible and tetracycline-repressible promoters (see, for example, U.S. Pat. Nos. 5,814,618 and 5,789,156, incorporated herein by reference).

Tissue-preferred promoters can be utilized. Tissue-preferred promoters, leaf-preferred promoters, root-preferred promoters, etc. as are known can be utilized and can be selected from those known in the art including, without limitation, those described in U.S. Pat. Nos. 5,837,876; 5,750,386; 5,633,363; 5,459,252; 5,401,836; 5,110,732; and 5,023,179, all of which are incorporated herein by reference.

In one embodiment, anther or pollen-preferred promoters may be used. For instance, while the RNA precursor nucleotide sequence may be operably linked to many possible promoters, it may be preferred to express the sequence with an anther preferred or pollen preferred promoter to prevent even low expression of the miRNA in other tissues of the plant.

“Seed-preferred” promoters include both “seed-specific” promoters (those promoters active during seed development such as promoters of seed storage proteins) as well as “seed-germinating” promoters (those promoters active during seed germination). Such seed-preferred promoters include, but are not limited to, Cim1 (cytokinin-induced message); cZ19B1 (maize 19 kDa zein); celA (cellulose synthase); gama-zein; Glob-1; bean-phaseolin; napin;-conglycinin; soybean lectin; cruciferin; maize 15 kDa zein; 22 kDa zein; 27 kDa zein; g-zein; waxy; shrunken 1; shrunken 2; globulin 1, etc.

The expression cassette can be provided with a plurality of restriction sites for insertion of the sequence(s) to be under the transcriptional regulation of the regulatory regions.

An expression cassette may contain one or more genes in addition to the modified miRNA precursor gene that can be co-transformed into the plant cell. For example, the expression cassette may additionally contain selectable marker genes. Alternatively, one or more additional gene(s) can be provided to the plant cell via additional expression cassette(s).

Additional sequence modifications as are known to enhance gene expression in a cellular host are encompassed. These include, without limitation, elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to other miRNA expressing genes of the host cell.

The expression cassettes may contain additional 5′ leader sequences in the construct. Such leader sequences can act to enhance translation. Translation leaders are known in the art and include: picornavirus leaders, for example, EMCV leader (Encephalomyocarditis 5′ noncoding region); potyvirus leaders, for example, TEV leader (Tobacco Etch Virus); MDMV leader (Maize Dwarf Mosaic Virus), and human immunoglobulin heavy-chain binding protein (BiP); untranslated leader from the coat protein miRNA of alfalfa mosaic virus (AMV RNA 4); tobacco mosaic virus leader (TMV); and maize chlorotic mottle virus leader (MCMV).

In preparing the expression cassette, the various fragments may be manipulated, so as to provide for the sequences in the proper orientation and, as appropriate, in the proper reading frame. Toward this end, adapters or linkers may be employed to join the fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Any means for introducing the expression cassette into a cell are encompassed by the present invention. In addition, while the cell may in one embodiment be a plant cell of the type of plant that is intended for use in forming the ingestible composition, this is not a requirement and the cell that is transformed can be a different type of plant cell or a different type of cell altogether, as long as the cell may be incorporated in a plant such that the miRNA expression product of the sequence is present in an ingestible portion of the plant.

Vectors may be used to incorporate the genetic sequence into a cell and express the miRNA products by various methods generally known in the art. Suitable vectors for expressing genes can include those that are self-replicating, capable of systemic infection in a host, and stable. Additionally, the vector can be capable of containing the nucleic acid sequences that are foreign to the native sequence of the vector. Transient expression systems may also be used.

In one embodiment, a hyper-expression vector can be utilized to deliver the modified miRNA precursor gene to a cell. Hyper-expression vectors have been shown to replicate in a broad range of edible plants and can enhance production by as much as 100-fold. For instance, transient expression technology can be utilized together with a hyper-expression vector in a variety of edible plants. By way of example, a pTRAc-based plasmid vector or a deconstructed plant viral replication vector such as the TRBO vector can be utilized to deliver the modified miRNA precursor gene to a plant cell. A hyper-expression vector can provide the expression of the modified miRNA precursor gene under the control of a strong promoter such as the constitutive cauliflower mosaic virus (CaMV) 35S promoter. A hyper-expression vector can be utilized in some embodiments to ensure that the accumulation of the miRNA expression product can be high enough in a plant transformed to include the plant cells to provide the miRNA expression product in therapeutically effective dose upon ingestion of the plant. However, it should be understood that hyper-expression is not required in the methods, and the amount of plant tissue ingested can be modified as necessary to obtain a therapeutically effective dosage.

FIG. 1 illustrates a pTRAc vector and two derivatives of the pTRAc vector as may be utilized in some embodiments. A pTRAc vector can afford enhanced transcriptional activity by providing scaffold attachment regions on either side of the modified miRNA precursor gene. The pTRAc vector includes a CaMV 35S promoter (P35SS) with duplicated transcriptional enhancer; CHS, chalcone synthase 5′ untranslated region; pA35S, CaMV 35S polyadenylation signal for foreign gene expression; SAR, scaffold attachment region of the tobacco Rb7 gene; LB and RB, the left and right borders for T-DNA integration; ColE1ori, origin of replication for E. coli; RK2ori, origin of replication for Agrobacterium; bla, ampicillin/carbenicillin-resistance bla gene; npt II, kanamycin-resistance npt II gene; Pnos and pAnos, promoter and polyadenylation signal of the nopaline synthase gene. An insert can be cloned into pTRAc between EcoRI and Xhol both of which are indicated in FIG. 1.

pTRAkc-rbcs1-cTP is a derivative of pTRAc with the chloroplast-transit peptide sequence of the potato rbcS1 gene. pTRAkc-ERH contains a plant codon-optimized signal-peptide sequence from the murine mAb24 heavy-chain gene and the his6 (SEQ ID NO: 15) and endoplasmic reticulum (ER) retention (SEKDEL (SEQ ID NO: 16)) sequences. The pTRAkc-rbcs1-cTP and pTRAkc-ERH vectors also include the npt II gene for kanamycin resistance in plants.

SEQ ID NO.: 1 provides the sequence downstream of the 35S promoter of the pTRAc vector of FIG. 1 including a pA35S terminator (in bold font) and a polyadenylation signal (in bold and underlined italics). SEQ ID NO.: 2 provides the Full sequence of a pTRAc vector with no nptll gene and SEQ ID NO.: 3 provides the full sequence of a pTRAc vector that has a nptll cassette within the T-DNA, which may be preferred in some embodiments in formation of stable transgenic plants.

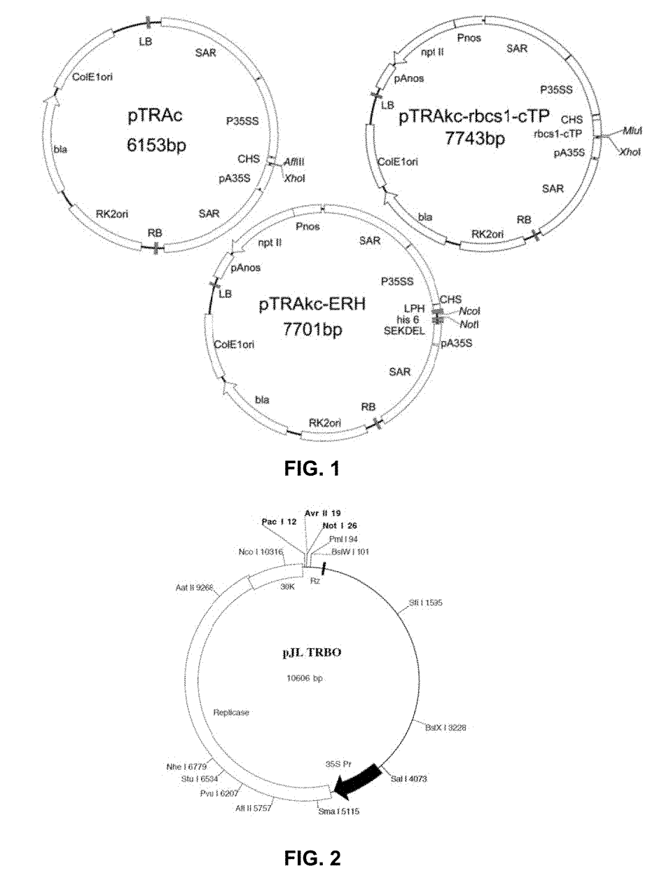

The deconstructed viral expression vector pJL TRBO (also known as PJL 48) is illustrated in FIG. 2. This binary plasmid vector is based on the single-stranded RNA plant virus tobacco mosaic virus (TMV), and can replicate in E. coli or Agrobacterium (the T-DNA borders are not shown in FIG. 2). The TRBO vector carries a DNA version of the TMV genome, but is missing the viral coat protein. Transcription of the TMV vector cDNA in the plant produces an RNA that can replicate, but does not produce virus particles and is not capable of long distance movement within the plant. Thus, this viral replicon system provides high level expression of inserted genes without concerns about spread of the virus. Genes of interest can be cloned into the Pacl-AvrII-Notl polylinker site as shown in SEQ ID NO.: 4.

There are two general strategies for expression of foreign genes in plants, either one of which can be utilized in expressing the therapeutic miRNA. In a stable transformation strategy, the gene of interest is incorporated into the plant genome, thereby creating stable transgenic plant lines. Such stably incorporated genes are inherited by successive generations of the transgenic line, and seeds from these plant lines can simply be planted for large scale production of the gene product of interest. However, generating and characterizing such lines can take months, and they can be subject to both transcriptional and post-transcriptional gene silencing in later generations.

In the transient expression strategy, the gene of interest is expressed transiently without incorporation into the genome. The transient expression strategy offers a rapid assay for determining effective combinations of vector and host plants. This strategy tends to result in higher expression than stable transformation and offers the advantage of a quick timeframe for production of the desired product, but does not produce heritable expression, so the process must be repeated for each batch of product. Transient expression systems are currently in widespread use for high-level production of beneficial proteins in plants, and this technique can be used in one embodiment for high level production of therapeutic miRNAs in plants.

Transient expression systems and stably transformed plant lines can both prove advantageous for commercial production of therapeutic miRNAs. For instance, transient expression studies may be preferred initially in selection of the best candidates for the longer term development of stable transgenic lines.

It should be understood, however, that a modified miRNA precursor gene can be transformed into plant cells using any suitable technology, such as a disarmed Ti-plasmid vector carried by Agrobacterium exploiting its natural gene transferability (see, e.g., U.S. Pat. No. 5,563,055, incorporated herein by reference); particle or microprojectile bombardment (see, e.g., U.S. Pat. Nos. 5,100,792 and 4,945,050 incorporated herein by reference); microinjection; electroporation; or other forms of direct DNA uptake (see, e.g., U.S. Pat. No. 4,684,611 incorporated herein by reference); liposome-mediated DNA uptake; the vortexing method; or other physical methods for the transformation of plant cells (see, e.g., U.S. Pat. Nos. 5,240,855, 5,322,783 and 5,324,646 incorporated herein by reference).

Agrobacterium transformation is widely used by those skilled in the art to transform dicotyledonous species. Agrobacterium mediated transformation has also emerged as a highly efficient transformation method in monocots

Microprojectile bombardment, electroporation or direct DNA uptake can be utilized in some embodiment, for instance where Agrobacterium is inefficient or ineffective. Alternatively, a combination of different techniques may be employed to enhance the efficiency of the transformation process, e.g., bombardment with Agrobacterium-coated microparticles or microprojectile bombardment to induce wounding followed by co-cultivation with Agrobacterium.

Following transformation, a plant may be regenerated, e.g., from single cells, callus tissue or leaf discs, as is standard in the art. Almost any plant can be entirely regenerated from cells, tissues, and organs of the plant. The transformed plants may then be grown, and either pollinated with the same transformed strain or different strains, and the resulting hybrid having expression of the therapeutic miRNA identified. Two or more generations may be grown to ensure that expression of the therapeutic miRNA characteristic is stably maintained and inherited (or not, as desired). In a stable transformation strategy, seeds can be harvested from the transformed plants to ensure expression of the therapeutic miRNA has been achieved.

The particular choice of a transformation technology will be determined by its efficiency to transform certain plant species as well as the experience and preference of the practitioner with a particular methodology of choice. It will be apparent to the skilled person that the particular choice of a transformation system to introduce nucleic acid into plant cells is not essential to or a limitation of the invention, nor is the choice of technique for plant regeneration.

Plants, seeds and plant parts that include the transformed plant cells or plant cells themselves in suspension culture or callus that include the therapeutic miRNA expression product can be utilized in forming an ingestible composition. Accordingly, in addition to a transformed plant, any clone of such a plant, seed, selfed or hybrid progeny and descendants, and any part of any of these, such as cuttings, seeds, etc. are encompassed herein. Any plant propagule, that is any part which may be used in reproduction or propagation, sexual or asexual, including cuttings, seed and so on is encompassed herein. Also encompassed is a plant that is a sexually or asexually propagated off-spring, clone or descendant of such a plant, or any part or propagule of said plant, off-spring, clone or descendant. Plant extracts and derivatives are also encompassed herein.

Any plant species capable of successful transformation and that can include the therapeutic miRNA expression product in ingestible tissue is encompassed herein. In one embodiment, Arabidopsis thaliana can be transformed with the modified miRNA precursor gene. This particular species is attractive for transformation because it is well characterized, easily transformed, edible, and allows for large scale production of the engineered miRNAs in environmental chambers. However, the methods and products are in no way limited to this species and other plant species of interest can include, but are not limited to, corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum, Nicotiana benthamiana), potato (Solanum tuberosum), tomato (Solanum lycopersicum), peanuts (Arachis hypogaea), cotton (Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, and other vegetable and fruits.

The miRNA expressing plants can include crop plants such as cereals and pulses, maize, wheat, potatoes, tapioca, rice, sorghum, millet, cassava, barley, pea, and other root, tuber, or seed crops. Examples of seed crops can include oil-seed rape, sugar beet, maize, sunflower, soybean, and sorghum. Other edible plants can include lettuce, endive, and vegetable brassicas including cabbage, broccoli, and cauliflower. The transformation methods may be applied to carrot, strawberry, sunflower, tomato and pepper. Grain plants that provide seeds of interest include oil-seed plants and leguminous plants. Seeds of interest include grain seeds, such as corn, wheat, barley, rice, sorghum, rye, etc. Oil seed plants include cotton, soybean, safflower, sunflower, Brassica, maize, alfalfa, palm, coconut, etc. Leguminous plants include beans and peas. Beans can include guar, locust bean, fenugreek, soybean, garden beans, cowpea, mungbean, lima bean, fava bean, lentils, chickpea, etc.

Following transformation and development of the plant tissue that incorporates the modified miRNA precursor gene, the plant can be capable of processing the expressed miRNA precursor via the miRNA biogenesis pathway to produce a therapeutic miRNA that is complementary to a particular target mRNA of a subject that ingests the plant. One or more components of the plant that carries the expressed miRNA can then be incorporated in an ingestible composition for delivery to a subject in need thereof via ingestion of the composition. For example plant tissue including, without limitation, root tissue, leaf tissue, fruit, seeds, or any other component of the transformed plant that can carry the therapeutic miRNA expression product of the modified miRNA precursor gene can be incorporated in an ingestible composition for delivery to a subject that carries the target of the therapeutic miRNA.

Plant tissue incorporating the therapeutic miRNA expression product can be orally administered, for example, with an inert diluent or with an assimilable edible carrier, or can be incorporated directly with the food of the diet. For example, the plant tissue can be incorporated with excipients and used in the form of ingestible, buccal tablets, troches, capsules, elixirs, suspensions, syrups, wafers, and the like. Such compositions and preparations can contain in one embodiment about 0.001% by weight or more of the of plant tissue. The percentage of the compositions and preparations may, of course, be varied and may conveniently be between about 0.001% to about 100% by weight of the plant tissue. For instance, in one embodiment, the plant tissue need not be combined with any other components in formation of an ingestible composition, and a subject can simply ingest the plant tissue alone. The amount of plant tissue in a therapeutically useful composition can be such that a suitable dosage of the therapeutic miRNA can be obtained by the subject.

When the form of the dosage unit is a tablet, troche, pill, capsule and the like, it may also contain one or more of the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. When the form of the dosage unit is a syrup or elixir, it may contain sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the form of the dosage unit may be a sustained-release, extended-release, or delayed-release preparation or formulation.

The plant tissue can be provided so as to provide the therapeutic miRNA in a therapeutically or prophylactically effective amount to the subject. In certain embodiments, such a compound or dosage unit is referred to as an active agent. As used herein, and unless otherwise indicated, the terms “treat,” “treating,” and “treatment” contemplate an action that occurs while a patient is suffering from a disease or disorder, that reduces the severity of one or more symptoms or effects of the disease or disorder, or a related disease or disorder. As used herein, and unless otherwise indicated, the terms “prevent,” “preventing,” and “prevention” contemplate an action that occurs before a patient begins to suffer from a disease or disorder, that prolongs the onset of, and/or inhibits or reduces the severity of, the disease or disorder. As used herein, and unless otherwise indicated, the terms “manage,” “managing,” and “management” encompass preventing, delaying, or reducing the severity of a recurrence of a disease or disorder in a patient who has already suffered from the disease or disorder. The terms encompass modulating the threshold, development, and/or duration of the disease or disorder, or changing the way that a patient responds to the disease or disorder.

As used herein, and unless otherwise specified, a “therapeutically effective amount” is an amount sufficient to provide any therapeutic benefit in the treatment or management of a disease or disorder, or to delay or minimize one or more symptoms associated with a disease or disorder. A therapeutically effective amount of a compound means an amount of the compound, alone or in combination with one or more other therapy and/or therapeutic agent, which provides any therapeutic benefit in the treatment or management of a disease or disorder, or related diseases or disorders. The term “therapeutically effective amount” can encompass an amount that cures a disease or disorder, improves or reduces a disease or disorder, reduces or avoids symptoms or causes of a disease or disorder, improves overall therapy, or enhances the therapeutic efficacy of another therapeutic agent.

As used herein, and unless otherwise specified, a “prophylactically effective amount” of a compound is an amount sufficient to prevent or delay the onset of a disease or disorder, or one or more symptoms associated with a disease or disorder, or prevent or delay its recurrence. A prophylactically effective amount of a compound means an amount of the compound, alone or in combination with one or more other treatment and/or prophylactic agent, which provides a prophylactic benefit in the prevention of a disease or disorder. The term “prophylactically effective amount” can encompass an amount that prevents a disease or disorder, improves overall prophylaxis, or enhances the prophylactic efficacy of another prophylactic agent.

Toxicity and therapeutic efficacy of the described compounds and compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, expressed as the ratio LD50/ED50. Compounds that exhibit large therapeutic indices are preferred. Compounds that exhibit toxic side effects may be used in certain embodiments, however, care should usually be taken to design delivery systems that target such compounds preferentially to the site of affected tissue, in order to minimize potential damage to uninfected cells and, thereby, reduce side effects.

Data obtained from cell culture assays and animal studies can be used in formulating a range of dosages for use in humans. In certain aspects of the present disclosure, the dosages of such compounds lie within a range of circulating concentrations that include the ED50 with little or no toxicity. The dosage may vary within this range depending on the dosage form employed and the route of administration utilized. For any compound used in the disclosed methods, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Plasma levels may be measured, for example, by high performance liquid chromatography.

When therapeutic treatment is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses. Additionally, the bioactive agent may be complexed with a variety of well-established compounds or structures that, for instance, enhance the stability of the bioactive agent, or otherwise enhance its pharmacological properties (e.g., increase in vivo half-life, reduce toxicity, etc.).

In certain embodiments of the present disclosure, the effective dose of the therapeutic miRNA or dosage unit can be in the range of about 10 mg/kg to about 0.01 mg/kg, about 10 mg/kg to about 0.1 mg/kg, about 10 mg/kg to about 1 mg/kg, or about 10 mg/kg to about 5 mg/kg in some embodiments.

Following ingestion, the therapeutic miRNA that is processed from the modified miRNA precursor can be capable of binding to a targeted human or animal mRNA and altering the expression of the protein it encodes. Depending upon the particular target and therapeutic effect desired, expression of a target sequence controlled via the miRNA pathway can be effectively silenced or partially reduced or alternatively increased. The term “increased expression” is intended to refer expression of a target nucleotide sequence being increased over endogenous levels of expression for the sequence. For example, in one embodiment the expression of the target nucleotide sequence can be increased about 25% or more, for instance from about 25% to about 50%, or from about 50% to about 100%, about 100% or greater, or about 1000% or greater in some embodiments.

The term “decreased expression” is intended to refer to expression of the target nucleotide sequence being decreased below endogenous levels of expression for the genes. For example, in one embodiment, expression of the target nucleotide sequence can be decreased about 25% or more, for instance from about 25% to about 50%, or from about 50% to about 100%.

The expression of the targeted protein can be assayed to determine that expression of the miRNA precursor results in altered gene expression in a subject. Expression levels may be assessed by determining the level of a gene product by any method known in the art including, but not limited to determining the levels of the RNA and protein encoded by a particular target gene. For genes that encode proteins, in one embodiment expression levels may be determined by quantifying the amount of the protein present in individual cells, in tissue, or in any portion of a subject. In those embodiments in which a target gene encodes a protein that has a known measurable activity, then activity levels may be measured to assess expression level of the gene.

Moreover, it should be understood that a method can include modulating the expression of one, two, or more target nucleotide sequences in a subject.

The present disclosure may be better understood with reference to the Examples, set forth below.

### EXAMPLE 1

C57BL/6J-ApcMin/J mice (Apc^{Min/+}) were purchased from Jackson Laboratories (Bar Harbor, Me., USA) and were bred and maintained at the Mouse Core Facility of the Center for Colon Cancer Research at the University of South Carolina (USC), Columbia, S.C. All aspects of the animal experiments were conducted in accordance with the guidelines and approval of the USC Institutional Animal Care and Use Committee. The Apc^{Min/+} mouse model of colon cancer is a genetic model of the disease. Apc^{Min/+} mice are heterozygous for the Min allele, a mutation of the Apc gene resulting in a truncated protein, and spontaneously develop 50 or more adenomas throughout the intestinal tract, making them a model of human disease such as FAP. These mice are relatively healthy and long lived, in contrast to orthotopic models of colon cancer, which quickly succumb to the disease. Apc^{Min/+} mice do not start to get sick until about 18 weeks of age, at which point they become anemic. They develop muscle wasting at about 20 weeks of age and typically die when about six months old. The treatment regimen ended before Apc^{Min/+} mice show any signs of illness.

Tumor suppressors miR-34a, miR-143, and miR-145 synthesized to have the 2′-O-methylation at the 3′-end, which is characteristic of miRNAs made in plants, were purchased from Integrated DNA Technologies. These three miRNAs are highly conserved in mammals and the sequences are the same in mice as in humans. These miRNAs have been validated as tumor suppressors in many studies. Total plant RNA for gavage was isolated from flash frozen Arabidopsis thaliana, ecotype Columbia, using TRIzol reagent (Life Technologies) according to the manufacturer's instructions, but with two additional ethanol precipitations to remove any traces of the reagent. Total plant RNA contains all high and low molecular weight RNA species present in the plant. Therefore, it contains all endogenous plant RNAs, including, for example, mRNAs, tRNAs and rRNA, as well as the entire set of endogenous plant miRNAs. In general, there is no homology between plant and animal miRNAs, although one bioinformatics study indicated that plants and animals share members of the miR854 family.

Five week old Apc^{Min/+} mice were divided into three treatment groups of seven mice each. The treatment groups corresponded to daily gavage with either 1) total plant RNA (from Arabidopsis thaliana, ecotype Columbia) spiked with a cocktail of tumor suppressor miRNAs commercially synthesized to have the 3′-methylation characteristic of plant miRNAs, 2) total plant RNA alone, or 3) water (all RNA preparations were dissolved in water).

Gavage with 150 μl of the preparations indicated above began when the mice were five weeks old and continued daily for four weeks. This time frame is a standard preventive regimen for experiments using Apc^{Min/+} mice. The daily doses in the RNA treatment groups corresponded to 61 μg total plant RNA alone or plus 4.5 to 7.4 μg (corresponding to 0.7 to 1.0 nmole) of each species of tumor suppressor miRNA. No weight loss was observed in any of the mice during the entire 28 days of treatment. Because one of the best signs of toxicity of therapeutic treatments has been loss of weight within 3-5 days of treatment, the absence of weight loss in the animals indicated that the treatments had no obvious toxicity. In addition, the animals did not develop anemia during the experiment, as indicated by normal (as opposed to pale) coloration of extremities, further arguing against toxic side effects.

Six hours after the last gavage treatment, mice were humanely sacrificed by cervical dislocation after administration of anesthesia using isoflurane by inhalation. The small and large intestines were removed, flushed with phosphate buffered saline, and sliced longitudinally. The small intestine was divided into four equal segments with the colon treated as the fifth segment. Sections for RNA isolation were flash frozen on dry ice, and segments for determination of tumor burden were fixed in 10% formalin and stained with 0.002% methylene blue. Tumors were counted under a dissecting microscope by a single highly experienced investigator, who was blinded to the treatments.

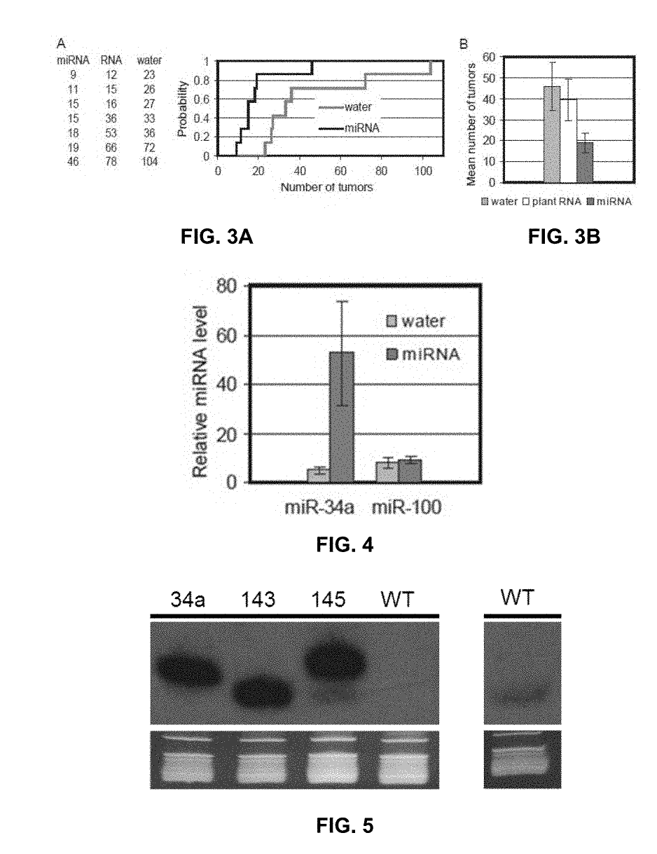

The K-S test was utilized to analyze the tumor number data because the test is sensitive to any differences in the distributions, including differences in shape, spread, or median and is relatively insensitive to outliers, thereby providing a robust test of whether the distributions are significantly different. The graphical presentation of the data in FIG. 3A, which shows the basis of the K-S test, enabled the observation that six out of seven of the mice in the tumor suppressor miRNA-treated group had fewer tumors than the mouse with the fewest tumors in the water-treated group. The one-sided K-S test was appropriate as the interest was in whether the number of tumors in the tumor suppressor miRNA-treated group was less than that in the water-treated group, not just whether the number of tumors was different in those two groups. The Mann-Whitney (M-W) directional test, another commonly used non-parametric statistic, gave a similar p-value for the difference between the miRNA-treated and water treated groups (M-W p=0.0075, K-S p=0.0058).

Flash frozen intestinal sections for RNA isolation were stored at −70° C. The frozen tissues were disrupted with a hand-held polytron at maximum speed in the presence of 10 ml of TRIzol reagent (Life Technologies) per gram of tissue, and total RNA was isolated according to manufacturer instructions.

Periodate treatment of the RNA from mouse tissue was used to specifically detect the administered miRNAs. In preparation for oxidation with sodium periodate, 40 of each total RNA sample from mouse intestine was brought up to 100 μl in sterile double-distilled water and then ethanol precipitated overnight at −20° C. in three volumes ethanol and 1/10 volume 3 M sodium acetate. Siliconized microcentrifuge tubes were used to minimize losses. Ethanol precipitates were spun down, washed in 75% ethanol, allowed to dry at room temperature, and resuspended in 80 μμl of an 8.3 pM solution (in sterile double-distilled water) of the spike-in normalization control miRNA, C. elegans miR-39 (2′-O-methylated at the 3′-end and purchased from Integrated DNA Technologies). Methylated C. elegans miR-39 was added to all samples before oxidation because endogenous RNA controls are all un-methylated and will be oxidized by periodate, rendering them useless for normalization. After being resuspended, each sample was divided into two equal (20 μg) portions, one to be oxidized and the other to serve as un-oxidized control. Quantitative real-time PCR of the un-oxidized control showed that the concentration of the spiked-in miR-39 relative to the endogenous normalization control RNU6-B was the same in all samples, indicating that use of the spiked-in, methylated miR-39 as the normalization control for the oxidized samples wouldn't introduce a systematic bias into the measurements.

Each 20 μg aliquot for oxidation was ethanol precipitated overnight as above and resuspended in 20 μl of borax/boric acid buffer pH 8.6 (0.06 M borax, 0.06 M boric acid). Then ⅛ volume (=2.5 μl) 200 mM sodium periodate was added to each, and the samples were incubated for one hour at room temperature in the dark. Next, 2 μl 100% glycerol was added to each, and incubation in the dark was continued for 40 minutes more. Finally, the samples were ethanol precipitated as above, two times, after which each was resuspended in 40 μl sterile double-distilled water. Resuspended RNA samples were stored frozen at −20° C.

The miScript-PCR system (Qiagen), including the miScript II RT Kit, miScript SYBR Green PCR Kit, and miScript primer assays was used for miR-34a, miR-143, miR-145, miR-100, and miR-39 according to the manufacturer's instructions. The reverse transcription (RT kit) reactions were performed using miScript HiSpec buffer and 1.5 μg RNA. The resulting cDNA was diluted 11-fold in sterile double-distilled water (the minimum amount specified in the kit protocol), aliquoted, and stored at −20° C. Quantitative real-time PCR reactions were performed in triplicate using an Applied Biosystems 7300 machine and the reaction and cycling conditions specified in the miScript-PCR protocol. 2.5 μl 11-fold diluted cDNA was used per reaction, which is the maximum amount of cDNA specified in the kit protocol. The normalization control quantitative real-time PCR reactions were always performed in the same run as the reactions for the miRNA of interest.

FIG. 3A shows the number of tumors for each mouse in the three different groups and a plot of those numbers for the miRNA-treated and water-treated groups. A one-sided K-S test showed a highly significant reduction in tumor burden in the miRNA-treated mice compared to the water-treated mice (p-value=0.0058). The K-S plot highlighted a striking feature of the data, which is that six of the seven mice in the tumor suppressor miRNA-treated group had fewer tumors than the mouse with the fewest tumors in the water-treated group. The tumor burden in the mice treated with total plant RNA alone was less than in the water-treated mice, suggesting that RNA alone may have a therapeutic effect. However, the two groups were not statistically different (p-value=0.28), and a larger sample size would be required to evaluate the therapeutic potential of plant RNA. FIG. 3B shows the average number of tumors in the three groups.

To determine the levels of administered tumor suppressor miRNAs in mouse intestine, the fact was exploited that the synthesized miRNAs used in the experiment, like plant-produced miRNAs, are methylated at the 2′ position of the ribose of the 3′ nucleotide, whereas endogenous mouse miRNAs are not. Periodate oxidation of unmethylated-miRNA breaks the bond between the 2′ and 3′ carbons of the 3′ terminal ribose, preventing subsequent in vitro polyadenylation. In contrast, methylated-miRNAs are resistant to periodate oxidation, providing a robust method to reduce the background of mouse miRNAs and thereby allowing specific quantitation of the orally administered methylated miRNAs. Periodate oxidation was used followed by polyadenylation, reverse transcription, and quantitative real-time PCR (RT-qPCR) to assay the level of miR-34a in intestinal RNA isolated from the mice. Relative miRNA concentrations were calculated from the RT-qPCR results, using the ΔΔCₜ method. This method is based on differences in the amplification curves of the reactions during the exponential phase of amplification, when starting miRNA concentrations can be accurately measured. The analysis showed with high statistical significance that the level of miR-34a in the miRNA-treated group was higher in the miRNA-treated mice than in the water-control mice (K-S test p-value=0.0082), with an average increase of about 10-fold (FIG. 4)

In contrast, the level of miR-100, an endogenous mouse miRNA that had about the same background level in the intestine as miR-34a, but was not fed to the mice, was not significantly different between the miRNA-treated and water-control groups (FIG. 4).

To rule out that the observed difference in miR-34a level between miR-treated and water-control groups was due to spurious amplification of unrelated molecules, the RT-qPCR products were analyzed at the end of the reactions using gel electrophoresis (FIG. 5). Because all the amplification curves had reached essentially the same plateau level at this point, the gel didn't reflect differences in starting concentrations of the miRNAs. However, it showed that a single product of the expected size was produced in all reactions, indicating that amplification was specific. Unfortunately, specific detection of the administered miR-143 and miR-145 in mouse intestine was not possible because the background of endogenous miR-143 and miR-145 after periodate oxidation was too high.

The results indicated that tumor suppressor miRNAs designed to mimic small RNAs produced in plants were taken up by the digestive tract of Apc^{Min/+} mice upon ingestion, as evidenced by their higher concentration in the miRNA-treated animals (FIG. 4), and were functional, as evidenced by the reduction in tumor burden (FIG. 3A and FIG. 3B).

### EXAMPLE 2

Three transgenic Arabidopsis thaliana lines expressing miRNA-34a, miRNA-143,and miRNA-145 were produced. The transgenic lines expressed the miRNAs at levels greater than that of the highly expressed endogenous plant miRNA, miR168a. The production technique used a natural plant miRNA precursor gene (miR319; ˜405 nt), as the starting structure but replaced the natural miRNA sequence with the desired miRNA sequence. Simultaneously, the natural, partially complementary miRNA* sequence was also replaced to maintain the original secondary structure of the precursor and ensure proper processing. The engineered miRNA gene was then cloned under control of a strong promoter and introduced into plants using standard transformation and regeneration techniques. The engineered miRNAs were essentially indistinguishable from endogenous plant miRNAs with regard to both biogenesis and function.

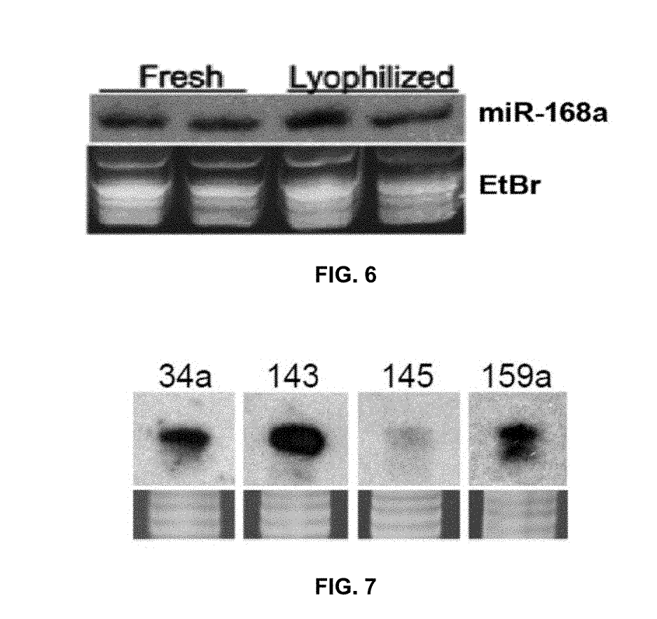

The stability of the miRNAs in lyophilized plant tissue was tested. The miRNA was stable, as shown by RNA gel blot analysis of RNA isolated from two fresh and two lyophilized plant tissue samples, using a probe specific for an endogenous plant miRNA (miR168a) (FIG. 6).

Apc^{Min/+} mice were fed a diet including 10% of the lyophilized transgenic A. thaliana tissue. The tumor burden was about two-fold lower in the treated mice compared to those fed a control diet (six mice in each group, p=0.045). The dosage of tumor suppressor miRNA provided was about 680 ng/day, compared to 23 μg/day of synthesized miRNA in Example 1, suggesting that delivery of miRNA in plant tissue was more effective than oral administration of synthesized miRNA.

Exosome-like vesicles (EVs) from the transgenic Arabidopsis were isolated using a standard protocol with some modification involving differential centrifugation of plant juice in a high-speed centrifuge (collecting the supernatant each time) and pelleting the last supernatant in an ultracentrifuge. RNA was extracted from these EVs and RNA gel blot analysis was used to detect the three tumor suppressor miRNAs as well as the abundant endogenous plant miRNA, miR159a (FIG. 7). Both miR-34a and miR-143 were present in amounts comparable to that of miR159a. MiR-145 was present at a much lower level, suggesting that there might be specificity in packaging into EVs. These data demonstrate that plant-produced mammalian tumor suppressor miRNAs, like natural plant miRNAs, can get packaged into a form taken up by the digestive tract.

While the EV preparations from the transgenic A. thaliana tissue had significant levels of miRNA, ultracentrifugation is not scalable. As such, a method involving precipitation by low-speed centrifugation in the presence of PEG 8,000 was evaluated. This method, using a commercial reagent (ExoQuick TC (System Biosciences)), produced EVs similar in quality to ultracentrifugation from animal biofluids, although it had not previously been applied to plants. In a preliminary experiment it was found that this method recovered 63% of miR143 expressed transiently in Nicotiana benthamiana leaves in just 10% of the total dry weight. In this procedure, leaf tissue from Nicotiana benthamiana was homogenized in phosphate buffered saline (PBS) at a ratio of 1:2 (w:w). The homogenate was centrifuged in a microcentrifuge at high speed (13,000 rpm) for 10 minutes. The supernatant was reserved and the pellet discarded. The supernatant was mixed with ExoQuick TC at a ratio of 5:1 (supernatant:ExoQuick TC) and incubated at 4° C. overnight. The mixture was centrifuged at 1500×g for 15 minutes at 4° C. The supernatant and pellet (EVs) were separated and miR143 in each were quantified separately. A duplicate sample of tissue was processed in this way and the pellet (EVs) from 1 gram of leaf tissue was freeze dried (lyophilized). The dry weight of the EV pellet was approximately 10% of the dry weight of 1 gram (wet weight) of leaf tissue.

### PROPHETIC EXAMPLE 1

A gene encoding an miRNA sequence can be designed using well-established techniques (see, e.g., Ossowski et al., 2008, Schwab et al., 2010). A natural plant miRNA precursor gene (for example, the Arabidopsis thaliana miR319 gene) can be used as the starting structure, but the natural plant miRNA and miRNA* sequences will be replaced with a desired miRNA and corresponding miRNA* sequences.

Replacement of both the miRNA and miRNA* sequences maintains the original secondary structure of the precursor and ensures proper processing by DCL1 to produce the correct miRNA. The sequence of the A. thaliana miR319 gene is shown in SEQ ID NO.: 5. The sequences in bold type are the miRNA* (the first bold sequence) and the miRNA (the second bold sequence). These will be replaced with sequences of the human miRNA miR-34a. The miR-34a miRNA* sequence (replacing the first bold sequence) is provided in SEQ ID NO.: 6. The miR-34a miRNA sequence (replacing the second bold sequence) is provided in SEQ ID NO.: 7.

Additional sequences to facilitate restriction digestion and cloning can be added to the 5′ and 3′ ends of the DNA sequence. The final designed sequence (SEQ ID NO.: 8) can then be chemically synthesized as a double-stranded DNA fragment. This can be accomplished by solid-phase synthesis of oligonucleotides from nucleoside phosphoramidites, followed by annealing, ligation and polymerase chain reaction (PCR) of the oligonucleotides to generate the desired double-stranded DNA fragment. There are many companies that produce synthetic DNA of specified sequence as a service. In SEQ ID NO.: 8, the sequences at the 5′ and 3′ ends allow for digestion by the enzymes EcoR I and Xba I, respectively.

Alternatively, sequences in the miR319 gene can be replaced by sequences of the human miR-143 miRNA (SEQ ID NO.: 9) and miRNA* (SEQ ID NO.: 10), resulting in SEQ ID NO.: 11, or of the human miR-145 miRNA (SEQ ID NO: 12) and miRNA* (SEQ ID NO.: 13), resulting in SEQ ID NO.: 14.

Following formation, the synthetic DNA fragment can be cut with the enzymes EcoR I and Xba I and then cloned by ligating with the binary plasmid expression vector pTRAkc (SEQ ID NO: 3). The resulting plasmid can be used for standard heat-shock transformation of chemically-competent E. coli. Transformants can be selected on Ampicillin and the cloned plasmid can be purified by standard “mini-prep” methods. The purified plasmid can then be transformed into Agrobacterium tumefaciens by electroporation. Transformants are selected on kanamycin. Several A. tumefaciens strains may be used, including GV3101 (pMP90RK), GV3101 (pMP90), LBA4404, EHA105 and C58. The resulting A. tumefaciens clone can be used for either stable or transient transformation of an edible plant species.

The aboveground parts of plants can be infiltrated using Agrobacterium tumefaciens carrying one of the miRNA expression vectors produced as described above, using standard vacuum infiltration methods (see, e.g., Leuzinger et al., 2013, Simmons et al., 2009). A strain of A. tumefaciens carrying the P19 suppressor of silencing can be co-infiltrated to maximize expression. Tissues can be analyzed at time points between three and twenty days after co-infiltration (or injection) for levels of the introduced miRNA. RNA can be isolated by a standard Trizol-based procedure and tumor suppressor miRNA accumulation can be determined by RT-qPCR analysis using primers specific for the miRNA of interest.

A. tumefaciens strains carrying pTRAkc-based miRNA expression vectors can also be used for stable transformation of plant tissue and regeneration of transgenic plants. A variety of protocols can be used, with different plant species optimally transformed by different protocols. In general this involves exposing plant tissue to A. tumefaciens carrying the plasmid of interest and placing the plant tissue onto a solid tissue culture medium containing nutrients and plant hormones along with an agent to select for transformed cells (in this example, kanamycin is used, but other selection agents, such as herbicides or plant hormones can be used if the appropriate selectable marker is present on the plasmid). Protocols for different edible plant species, including, but not limited to, Brassica (broccoli, cauliflower, cabbage, kale, etc.), carrot, and tomato can be used as found in the literature.

### PROPHETIC EXAMPLE 2

A construct for expressing a 21-nt version of miR-145 that has been formed (2 nt were deleted from the 3′-end, which should not affect specificity) will be used for stable and transient plant transformation. RNA gel blot analysis will be used to determine if the resulting transgenic plants package the 21-nt version of miR-145 into exosomes.

The engineered miRNA genes (including the shorter miR-145 gene) will be cloned into pTRAkc and pTRBO. The new constructs will be introduced into A. tumefaciens GV3101 and transient expression initiated by agro-infiltration of N. benthamiana. Whole N. benthamiana plants, inverted and submerged into an aqueous agrobacterium suspension, will be subjected to vacuum followed by slow vacuum release to draw the bacterial suspension into the spongy leaves. Following infiltration, plants will be grown for up to 8 days in a controlled environment. Typically, mRNA expression peaks 3-4 days following agro-infiltration. RNA will be isolated by a standard Trizol-based procedure. Tumor suppressor miRNA accumulation, both in whole tissue and isolated EVs, will be determined at a range of time points by RT-qPCR analysis. The vectors and time points that result in the highest levels of each tumor suppressor miRNA will be used to produce and freeze-dry EVs for use in animal feeding experiments.

Methods for isolation and preservation of plant exosomes will be developed to supply feeding experiments. Specifically, EVs will be isolated from transgenic plants that produce tumor suppressor miRNAs and the EVs formulated into mouse chow in a way that maintains EV integrity. While N. benthamiana contains nicotine and is not palatable directly, nicotine is in the soluble fraction so will not be recovered with EVs. Any material used for animal feeding will be tested for residual nicotine.

Quantitative PCR (q-PCR) analysis will be used to assay recovery of miR-34a, miR-143, miR-145 and the endogenous miR-159 following precipitation with a range of PEG 8,000 concentrations. Once scalable high yield EV purification is developed, RNA libraries will be prepared and next-generation RNA sequencing will be performed using standard procedures. These sequencing results will help determine whether there is specificity in packaging of miRNAs into EVs or whether their abundance in EVs is simply a function of their abundance in the plant tissue. This information will be important for determining whether special measures might be necessary to ensure packaging of certain therapeutic miRNAs into EVs. EVs will be isolated (as was done with whole tissue; above) to prepare them for formulation into mouse feed.

### PROPHETIC EXAMPLE 3

While the initial test of PEG precipitation to isolate EVs resulted in recovery of ˜63% of miR-143 (the rest is in the soluble fraction of juice), there may be differences in the biological activity or bioavailability of miRNA in the EV and soluble fractions. By simply dialyzing or diafiltering juice to remove nicotine, then lyophilizing, both fractions will be recovered in a form that can be formulated into mouse chow. As with the EV preparations, miRNA recovery and integrity will be analyzed by q-PCR and northern blotting.

The amount of each tumor suppressor miRNA in the plant material will be quantified by RT-qPCR analysis prior to formulation. Tumor number and size, as well as levels of administered miRNAs in mouse tissues will be assayed at the end of the treatment regimens to determine efficacy of feeding the bioengineered plants as a chemopreventive strategy.

There will be five experimental groups of 12 mice each, corresponding to two therapeutic and three control diets. The diets will include:

1) Therapeutic EV diet—a standard healthy mouse diet base into which lyophilized EVs from plants producing miR-34a, miR-143, and miR-145 have been incorporated. This diet will also contain natural plant miRNAs.

2) Control EV diet—exactly like the therapeutic EV diet, except using EVs from non-transgenic plant tissue. This diet is to check for a possible chemopreventive activity of plant EVs.

3) Therapeutic juice diet—the same standard healthy mouse diet base as above into which lyophilized juice from plants producing miR-34a, miR-143, and miR-145 has been incorporated. This diet will also contain natural plant miRNAs.

4) Control juice diet—exactly like the therapeutic juice diet, except using juice from non-transgenic plant tissue

5) Control diet—comprised of purified components with the same nutritional and caloric content as the first four diets, but containing neither tumor suppressor miRNAs nor natural plant miRNAs.

Feeding of these custom diets will start at four weeks of age and continue for six weeks, a standard preventive regimen time frame for experiments using Apc^{Min/+} mice. To assess the effect of the treatments on the overall health of the mice, their weights will be monitored daily, and blood samples will be analyzed for various blood parameters before starting the regimen, weekly thereafter, and just prior to sacrifice. Based on calculations, a sample size of at least 12 mice per group should be sufficient to satisfy power requirements. This power analysis is based on the assumption that the tumor counts are Poisson distributed for each group, an assumption that will need to be validated once the data are in hand.

The mice will be sacrificed and intestinal tissues collected for RNA analysis and assessment of tumor number and size. To improve the chances of detecting the administered miR-143/145, RNA will be isolated from several different intestinal regions and cell types, some of which might be enriched for ingested miRNAs. These samples will include tumors, tissue remaining after tumor excision, mucosal membranes, and an intact section of the intestine. After samples are taken, the remaining intestinal tissue will be fixed in 10% formalin and stained with 0.002% methylene blue to allow counting of any residual tumors. All tumor counting will be done under a dissecting microscope by a single experienced investigator, who will be blinded to the treatments.

RNA will be isolated using a Trizol-based procedure, and levels of the plant-synthesized miR-34a, miR-143 and miR-145 measured by qPCR using specific stem-loop primers to generate cDNA. Periodate oxidation will be used to specifically quantitate the methylated (i.e., plant produced) miRNAs.

The efficacy of additional tumor suppressor miRNAs will also be evaluated. For example, both miR-126 and miR-20b are downregulated in FAP adenomas, and could be evaluated for therapeutic effect.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A cell comprising an exogenous genetic sequence, the exogenous genetic sequence encoding a therapeutic miRNA, the therapeutic miRNA being configured to modulate a target nucleotide sequence, wherein the cell is free of the target nucleotide sequence.

2. The cell of claim 1, wherein the cell is a plant cell.

3. The plant cell of claim 2, wherein the plant cell is a component of or derived from an ingestible plant.

4. The cell of claim 1, wherein the exogenous genetic sequence is a component of a modified miRNA precursor gene, the modified miRNA precursor gene being derived from a starting miRNA precursor gene that is an endogenous miRNA precursor gene of the cell.

5. The cell of claim 1, wherein the exogenous genetic sequence is a component of a modified miRNA precursor gene, the modified miRNA precursor gene being derived from a starting miRNA precursor gene that is exogenous to the cell.

6. The cell of claim 1, wherein the therapeutic miRNA expression product of the exogenous genetic sequence is completely complementary to the target nucleotide sequence.

7. The cell of claim 1, wherein the therapeutic miRNA expression product of the exogenous genetic sequence exhibits from about 1 to about 6 nucleotide mismatches with the target nucleotide sequence.

8. The cell of claim 1, wherein the therapeutic miRNA expression product of the exogenous genetic sequence alters the production, processing, stability or translation of the target nucleotide sequence.

9. The cell of claim 1, wherein the target nucleotide sequence is an endogenous sequence of an animal or human.

10. The cell of claim 1, wherein the therapeutic miRNA is a tumor suppressor miRNA.

11. The cell of claim 8, wherein the tumor suppressor miRNA is miR-34a, miR-143, or miR-145.

12. The cell of claim 1, wherein the exogenous genetic sequence is a component of a hyper-expression vector.

13. Ingestible plant tissue comprising the cell of claim 1 and/or the miRNA expression product of the exogenous genetic sequence of the cell of claim 1.

14. A method for forming an ingestible therapeutic composition, the method comprising:
forming a modified miRNA precursor gene to include a genetic sequence encoding a therapeutic miRNA that is configured to modulate a target nucleotide sequence;
introducing the modified miRNA precursor gene into a cell, wherein the genetic sequence encoding the therapeutic miRNA is exogenous to the cell and wherein the cell is free of the target nucleotide sequence;
generating plant tissue that includes the cell and/or the therapeutic miRNA expression product of the genetic sequence; and
incorporating the plant tissue into an ingestible composition.

15. The method of claim 14, wherein the step of forming the modified miRNA precursor gene includes replacing an miRNA encoding genetic sequence and an miRNA* sequence associated therewith of a starting miRNA precursor gene with the genetic sequence encoding the therapeutic miRNA and an miRNA* associated therewith.

16. The method of claim 15, wherein the starting miRNA precursor gene is endogenous to the cell.

17. The method of claim 15, wherein the starting miRNA precursor gene is exogenous to the cell.

18. The method of claim 14, wherein the modified miRNA precursor gene is introduced into the cell in conjunction with a hyper-expression vector.

19. The method of claim 14, wherein the modified miRNA precursor is introduced into the cell according to a stable transformation strategy.

20. The method of claim 14, wherein the modified miRNA precursor is introduced into the cell according to a transient expression strategy.

21. The method of claim 14, wherein the ingestible composition consists of the plant tissue.

22. The method of claim 14, wherein the ingestible composition includes the plant tissue in an amount of from about 0.001% to about 100% of the plant tissue.

23. A method for delivery of a therapeutic miRNA to a subject in need thereof, the method including providing to the subject an ingestible composition comprising plant tissue from a plant, the plant including a genetic sequence encoding the therapeutic miRNA, the genetic sequence being exogenous to the plant, the plant tissue comprising the genetic sequence and/or the therapeutic miRNA expression product of the genetic sequence, the therapeutic miRNA modulating a target nucleotide sequence that is absent from the plant and that is carried by the subject.

24. The method of claim 23, wherein the subject is suffering from cancer.

25. The method of claim 24, wherein the subject is suffering from a colon cancer or an inflammatory bowel disease.

26. The method of claim 23, wherein the therapeutic miRNA is a mammalian tumor suppressor miRNA.

27. The method of claim 23, wherein the ingestible composition consists of the plant tissue.
